(19) United States
(12) Patent Application Publication
(10) Pub. No.: US 2016/0015920 A1
(43) Pub. Date: Jan. 21, 2016

(54) BIOMIMETIC ARTIFICIAL SECURED AIRWAY

(71) Applicant: Eric Robert WEBER, Paris, KY (US)

(72) Inventor: Eric Robert Weber, Groton, MA (US)

(21) Appl. No.: 14/114,420

(22) PCT Filed: May 11, 2012
(86) PCT No.: PCT/IB12/03140

§ 371 Date: Nov. 20, 2013

Related U.S. Application Data
(60) Provisional application No. 61/485,221, filed on May 12, 2011.

(51) Int. Cl. A61M 16/04

(52) U.S. Cl. CPC A61M 16/047; A61M 16/0463; A61M 16/0434

(57) ABSTRACT

Disclosed herein is a biomimetic artificial secured airway for use with a patient ventilating system that reduces the incidence of ventilator-associated patient infections when compared with the infection rate attributable to existing endotracheal and tracheostomy tubes.

This utility patent application is being submitted as a PCT application with the USPTO as the chosen receiving office and claims priority from provisional application 61/485,221 filed by the inventor in the USPTO on 12 May 2011, the entire contents of which are incorporated herein by reference.

### FIELD OF THE INVENTION

The instant invention relates to a secured artificial airway for mechanically ventilating a medical patient, whether for long-term ventilation support or short-term ventilation, and whether in a critical care environment like an Intensive Care Unit (ICU) or in a sub-acute or home care situation. More specifically the invention is related to providing a device that closely approximates the native airway physiology and reduces associated infections. It includes a secured airway from the trachea to the exterior of the body, a method of drainage and secretion removal, and controller that regulates the actuation of short fibers on the surface of the airway and removal of accumulated debris and secretions.

### REFERENCES CITED

| Document Number | Title | Filing date |
|---|---|---|
| EP1120124 | Ventilator | Dec. 5, 2000 |
| EP0982043 | Ventilator | Aug. 9, 1999 |
| U.S. Pat. No. 7,219,666 | Ventilator | Jul. 15, 2004 |
| EP1179354 | Ventilator | Jun. 15, 2001 |
| U.S. Pat. No. 6,516,800 | Neonatal patient ventilator | Aug. 25, 2000 |
| | circuit | |
| U.S. Pat. No. 6,369,838 | Graphic user interface for a | May 19, 1999 |
| | patient ventilator | |
| US20090194110 | Mechanical ventilator | Jan. 30, 2009 |
| U.S. Pat. No. 7,017,574 | System for automatically | Nov. 17, 2003 |
| | weaning a patient from a | |
| | ventilator, and method thereof | |
| EP1767235 | Ventilator | Sep. 18, 2006 |
| U.S. Pat. No. 6,082,357 | Mechanical ventilator | Mar. 25, 1998 |
| US20080308104 | Ventilator | Jun. 9, 2005 |
| US20090071478 | Ventilator | Sep. 17, 2008 |
| U.S. Pat. No. 6,910,480 | Patient-ventilator | Sep. 14, 2000 |
| | synchronization using dual | |
| | phase sensors | |
| U.S. Pat. No. 6,547,825 | Artificial trachea | Apr. 3, 2002 |

EP1120124 discloses and claims a ventilator for respiratory care, intended for connection to a patient, stating further that needless alarms, caused by a patient's attempts to speak and needless suffering because of the patient's inability to trigger an alarm, are some of the problems occurring. These problems are solved according to the invention when a sound detector is devised and arranged to detect and identify sounds made by the patient, preferably intentionally, and a control unit is operatively connected to the sound detector and devised to act on at least one function in the ventilator on the basis of sounds identified by the sound detector

EP0982043 discloses and claims a ventilator designed for connection to a patient to provide assisted breathing, comprising a gas flow generator, a pressure meter, a flow meter and a control unit, devised to determine a gas pressure, on the basis of a pre-set tidal volume for the patient and measurement signals from the pressure meter and flow meter, for each breath and to regulate the gas flow generator so it generates the gas pressure. Further claimed is an improved breathing mode, better tailored to the patient, achieved when the control unit is also devised to determine the gas pressure to be generated by the gas flow generator on the basis of mechanical resistances and a variable corresponding to the aggregate effect of the resistance and elastance of the lungs.

U.S. Pat. No. 7,219,666 discloses and claims a ventilator which has a compact block made of plastic or metal and includes an electrical computer interface, in which rigid pipes and a gas supply container are integrated, resulting in a more compact device than conventional superstructures and improved therapy gas delivery and nebulization.

EP1179354 discloses and claims a ventilator consisting of an inspiratory unit and an expiratory valve for regulating a flow of breathing gas and a control unit for controlling the inspiratory unit and the expiratory valve. Further, in order to facilitate the opening of collapsed alveoli in the lungs, the control unit is devised to control the inspiratory unit and the expiratory valve in generating a recruitment phase with an elevated basic pressure for the flow of breathing gas upon which a number of breaths is superimposed at a faster breathing rate.

U.S. Pat. No. 6,516,800 discloses and claims a disposable neonatal patient ventilator circuit for an automatic ventilator. All components that are exposed to contamination from the patient are packaged together in a disposable unit external to the automatic ventilator. The automatic ventilator has a receptacle with a pressurized breathable gas supply port, a breathing control circuit port, and preferably a patient monitoring port. The disposable neonatal resuscitator unit matches the ports on the automatic ventilator with a removable plug having a pressurized breathable gas supply connector mating the gas supply port of the receptacle.

U.S. Pat. No. 6,369,838 discloses and claims a ventilation control system for controlling the ventilation of a patient. The ventilation control system utilizes a user-friendly user interface for the display of patient data and ventilator status, as well as for entering values for ventilation settings to be used to control the ventilator.

US20090194110 discloses and claims an entrainment-based mechanical ventilator that may be initially set to approximate the patient's spontaneous respiratory frequency, enabling the patient to entrain to the ventilator if the ventilator frequency matches the patient's spontaneous breathing frequency or is within a reasonable range from it. If the preset frequency of the ventilator is set too high or too low from the spontaneous frequency, the patient will fall out of phase and “fight” the ventilator. The ventilator phase relative to the patient's breathing effort indicates the difference between the ventilator frequency and spontaneous frequency. Based on the phase difference, a closed-loop control mechanism may continuously adjust the ventilator frequency to match the patient breathing frequency until the phase shift is within a preset limit.

U.S. Pat. No. 7,017,574 discloses and claims a medical mechanical ventilator device adapted for use in weaning a patient from mechanical ventilation. In one embodiment, the device measures the patient's minute volume, breath frequency, and detects a patient's spontaneous breath. The device compares the patient's minute volume and the patient breath rate to a predetermined minute volume and a predetermined breath rate entered by a clinician. In a pressure support mode, the device decreases patient pressure support level if the patient's spontaneous breathing rate falls within the predetermined range of breathing and the patient's minute volume exceeds the predetermined minute volume.

EP1767235 discloses and claims a ventilator for ventilating a patient, the ventilator having the means built-in for carrying out a determination of the functional residual capacity of the patient using an inert gas wash in/wash out technique. Accordingly, the ventilator operates to alter the inert gas content of breathing gases provided to the patient.

U.S. Pat. No. 6,082,357 discloses and claims a mechanical ventilator for ventilating a patient that employs an air displacement member mounted for oscillating motion in a chamber, preferably the chamber is semi-cylindrical and the air displacement member is a vane mounted for oscillating movement about an axis of rotation such that a free outer edge surface of the vane is maintained in closely spaced apart relationship with the interior wall of the chamber throughout the oscillating. The ventilator permits significant variation in the ventilation flow waveform.

US20080308104 discloses and claims a ventilator, intended to be connected to a patient for breathing therapy, which has a control unit having an input for receiving EMG signals from an EMG detector and an output for an EMG based control signal and a pneumatic unit for generating breathing gas flows dependent on the EMG based control signal is described. The ventilator has a detector for determining a parameter related to breathing dynamics for the patient, the detector being connected to the control unit and control unit controlling the pneumatic unit dependent on the parameter related to breathing dynamics in the case of loss of EMG signals at the input.

US20090071478 discloses and claims a ventilator to replace or supplement a patient's breathing includes a control valve in the form of a proportional obstacle valve (POV) to provide improved air flow control and ventilator operation reliability. The POV includes an inlet, an outlet and a bypass. A stopcock controlled by a stepper motor directs the flow of air through the bypass and outlet permitting the turbine to operate a constant RPM yet allowing control of the airflow to a patient. The ventilator also includes inhalation and exhalation valve assemblies which improve air flow control and are easy to manufacture. The inhalation valve includes an orifice disk to allow pressure sensors to move accurately measure air flow. The exhalation valve assembly includes wings to reduce turbulence and enhance sensor accuracy. The exhalation valve assembly is arranged to have warm air from cooling the turbine blow over the assembly to reduce the possibility of condensation forming therein. The ventilator also includes an improved power supply with redundant sources of power.

U.S. Pat. No. 6,910,480 discloses and claims an improved ventilator which delivers ventilation support that is synchronized with the phase of the patient's respiratory efforts and guarantees a targeted minimum ventilation. Improved synchronization is achieved through an instantaneous respiratory phase determination process based upon measured respiratory airflow as well as measured respiratory effort using an effort sensor accessory, preferably a suprasternal notch sensor.

U.S. Pat. No. 6,547,825 discloses and claims an artificial trachea that is able to be used safely in the clinical application, and especially an artificial trachea for the intra-thoracic trachea, and the artificial trachea comprises a polypropylene mesh tube for the base material, around the outer periphery of which a polypropylene filamentous stent is wound in a spiral shape, an amorphous collagen thin layer formed on the surface of the base material, and a fine fibrous collagen layer formed on the inner and outer surface of the amorphous collagen thin layer, to which thermal dehydration crosslinking is performed.

### BACKGROUND OF THE INVENTION

A medical mechanical ventilation system consists of a mechanical ventilator, a ventilator circuit that may include filters or humidifiers, and a secured airway, either through the nasal or oral cavities or through a surgically inserted tracheostomy opening into the trachea. The mechanical ventilator is a mechanical pump for moving air or gas through the system. The ventilator circuit provides a conduit for the air to move from the mechanical pump into the artificial airway and into the lungs. The secured airway is most commonly a PVC endotracheal tube, but may be an alternate form of nasotracheal or orotracheal airway or tracheostomy tube.

Mechanical ventilators can operate on a set cycle of inspiratory and expiratory pressures and volumes, and may include an occasional increase in pressure to open closed alveoli. The ventilator circuits are generally composed of a plastic tube with ports for sensors and medication administration. Filters or humidifiers placed as part of the ventilator circuit involve air moving through a membrane and introduction of fluid or humidified air to the circuit space.

Patients who are undergoing ventilator therapy may have an endotracheal tube placed to maintain the airway under mechanical ventilation. The artificial airway consists of a polyvinyl chloride tube that introduced through the oral cavity through the pharynx and larynx and terminates in the trachea below the larynx. The tube includes an inflatable cuff that is inflated to sufficient pressure to provide a seal at the tracheal end, and the distal end of the tube is secured at the mouth.

Artificial cilia formed from long chains of spherical superparamagnetic particles which self-assemble in a microfluidic device are known. Until this invention, however, no artificial ciliary capability appropriate for medical applications had been developed.

Although there have been recent advancements in structured antimicrobial and antibiotic therapy, nursing protocols, patient positioning, and endotracheal tube design, ventilator-associated infection continues to be a significant clinical problem with severe implications for patients with an already critical underlying co-morbidity factor that necessitates mechanical ventilation. The endotracheal tube acts as a conduit for infectious agents because it bypasses the host defense system of the trachea and provides a reservoir for bacteria to colonize where they exhibit increased resistance to antimicrobial agents.

### DETAILED DESCRIPTION OF THE INVENTION

One skilled in the art knows that despite recent improvements in medical mechanical ventilation systems, there has not been a concomitant decrease in the incidence of ventilator associated infection. This lack of improvement regarding the incidence of infection most likely results from the lack of previous ventilator system designs to include a biomimetic artificial airway designed to replicate or replace the native host immune system bypassed by the artificial system. The instant invention will overcome this shortcoming by providing a biomimetic solution to maintenance of an artificial secured airway.

The instant invention consists essentially of a secured airway tube that has an internal surface modification that approximates cilia and ciliary movement with a drain for removing biofilm and particles cleared from the airway by the ciliary action, as well as an electronic controller that varies the cycles of actuation of fibers on the interior surface of the airway and controls drainage and suction within the device. It will be readily understood by those skilled in the art that the heretofore unprecedented feature of this biomimetic airway, the ability to mimic the human body's natural airway clearing function, has the potential to decrease the occurrence of hospital-acquired infections, a major threat to public health as identified by the Centers for Disease Control and Prevention. As a successful preventive intervention, the instant invention will reduce reliance on antibiotic agents to treat infections and will therefore decrease the development of antibiotic resistant respiratory pathogen strains. The net result will be a healthier hospital environment both for patients and providers.

Additionally, this artificial cilia technology offers the potential for use as a cross-discipline platform for future innovation in the life sciences, being used for example in fabricating replacement devices for other tissues having naturally occurring cilia, including but not limited to the lining of the gastrointestinal system.

### BRIEF DESCRIPTION OF THE DRAWINGS

FIG. 1 is an overall view of the invention.

FIG. 2 shows a longitudinal section of the interior surface of the invention.

FIG. 3 shows a longitudinal section of the interior surface of the invention at two different time points.

### DETAILED DESCRIPTION OF THE DRAWINGS

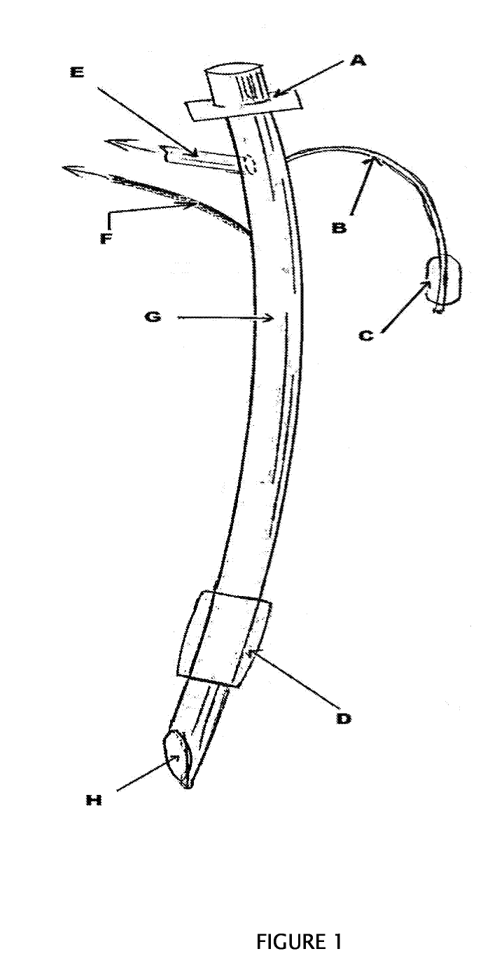

FIG. 1 is an overall outside view of the biomimetic artificial airway. Feature A is a universal adapter that connects the tube to standard airway circuitry. Feature B is the tubing of the air injection port that inflates the tracheal cuff. Feature C is a small balloon for optimizing cuff inflation. Feature D is a tracheal cuff balloon. Feature E is a drainage port that connects to suction or other drainage. Feature F is a wire bundle that connects to the electronic controller. Feature G is the main body of the tube. Feature H is the beveled tracheal end of the tube that includes a Murphy eye.

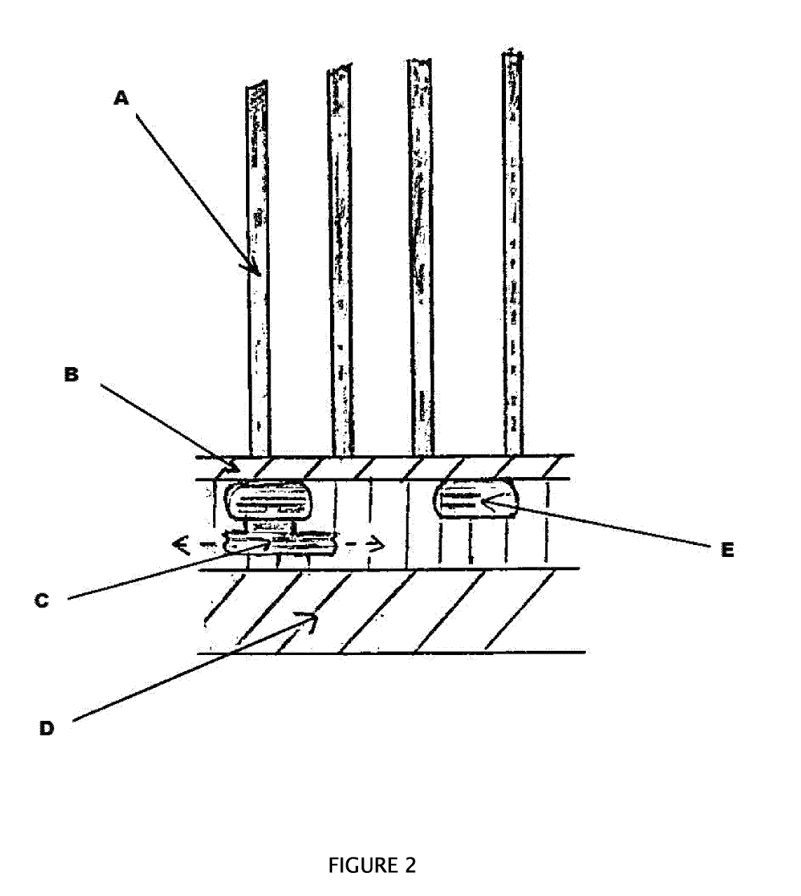

FIG. 2 illustrates a longitudinal section of the interior surface of the invention (where left and right are the long axis of the tube). Feature A is short fiber with the free end in the lumen of the airway and the other end attached to the body of the device. Feature B is a thin film for attachment of the fiber to the body of the device. Feature C is a longitudinal conductive circuit that connects circumferential circuits to the wire bundle leaving the device and to other circumferential circuits. Feature D is the bulk tube and may contain an air-injection passage from the air-injection port to the inflatable tracheal cuff balloon. Feature E is a circumferential conductive circuit.

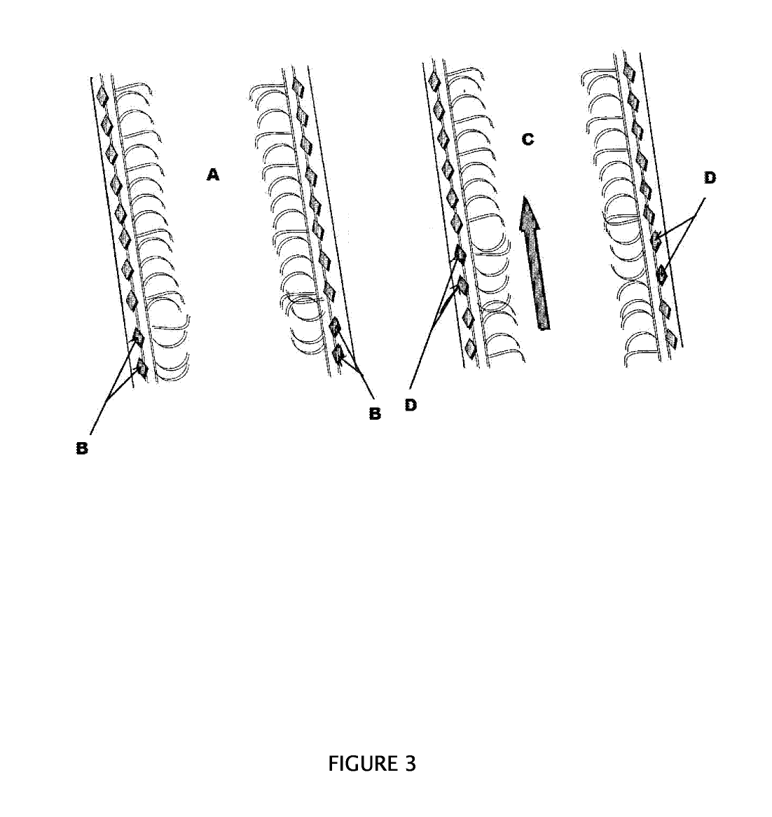

FIG. 3 illustrates a longitudinal section of the interior surface of the invention at two different time points (where up and down are the long axis of the tube). Features A and C are the lumen of the airway. Feature B is energized circumferential circuits in the tube at one time point while feature D is the energizing of sequential circumferential circuits in the same tube at a second time point, with the thick arrow showing a corresponding longitudinal wave in the interior fibers.

What is claimed is:

1. A biomimetic artificial secured airway that reduces the incidence of ventilator-associated patient infections when compared with the infection rates attributable to the use of endotracheal and tracheostomy tubes presently available.

2. An airway as in claim 1 further comprising the means to enable its interior surface to emulate the human body's natural defense mechanism against respiratory system infections by artificial cilia-like longitudinal wave action to move debris and pathogenic bacteria out of the patient's respiratory tract.

3. An airway as in claim 2 further comprising a means to empty the collected debris and pathogenic bacteria from the secured airway structure.
